(19) United States
(12) Patent Application Publication
(10) Pub. No.: US 2008/0188940 A1
(43) Pub. Date: Aug. 7, 2008

(54) Spinal Implant

(75) Inventors: Robert C. Cohen, Rockaway Township, NJ (US); Chistopher J. Valois, Champlin, MN (US); David A. Hanson, Urbandale, IA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER SPINE, INC., Minneapolis, MN (US)

(21) Appl. No.: 11/767,673

(22) Filed: Jun. 25, 2007

Related U.S. Application Data
(62) Division of application No. 29/276,676, filed on Feb. 1, 2007.
Application No. 29/276,676 is a division of application No. 29/277,221, filed on Feb. 19, 2007.
Application No. 29/277,221 is a division of application No. 29/277,352, filed on Feb. 22, 2007.

(51) Int. Cl. A61F 2/44

(52) U.S. Cl. 623/17.16

(57) ABSTRACT

A spinal implant is provided that includes a body having at least one piece of cortical bone. The body has a tapered leading end, a trailing end and first and second sides. The body also includes superior and inferior surfaces that are inclined relative to one another. A first plurality of grooves is formed in the superior surface and a second plurality of the grooves is formed in the inferior surface. Each groove of the first and second pluralities of the grooves has first and second opposing faces converging toward and intersecting one another. Each groove has a maximum cross-sectional width. Each adjacent pair of the grooves of the first and second pluralities of the grooves is separated by a generally planar portion of the superior and inferior surfaces, respectively. Each of the generally planar portions has a width that is equal to or greater than the maximum cross-sectional width of each of the grooves of the respective adjacent pair of the grooves.

### CROSS REFERENCE

This application claims priority to U.S. Design patent application Ser. No. 29/276,676, “Spinal Implant”, filed Feb. 1, 2007; U.S. Design patent application Ser. No. 29/277,221, “Spinal Implant”, filed Feb. 19, 2007; and U.S. Design patent application Ser. No. 29/277,352, “Spinal Implant”, filed Feb. 22, 2007, each disclosure of which is expressly incorporated by reference herein in its entirety.

### FIELD OF THE INVENTION

The present invention relates generally to skeletal implants. More particularly, the present invention relates to implants for stabilizing intervertebral joints.

### BACKGROUND OF THE INVENTION

Chronic back problems cause pain and disability for a large segment of the population. In many cases, chronic back problems are caused by intervertebral disc disease. When an intervertebral disc is diseased, the vertebrae between which the disc is positioned may be inadequately supported, resulting in persistent pain. Stabilization and/or arthrodesis of the intervertebral joint can reduce the pain and debilitating effects associated with disc disease.

Spinal stabilization systems and procedures have been developed to stabilize diseased intervertebral joints and, in some cases, to fuse the vertebrae that are adjacent the diseased joint space. Most fusion techniques include removing some or all of the disc material from the affected joint, and stabilizing the joint by inserting an implant (e.g., a bone graft or other material to facilitate fusion of the vertebrae) in the cleaned intervertebral space.

Spinal implants can be inserted into the intervertebral space through an anterior approach, a lateral (transverse) approach, a posterior approach, or postero-lateral approach. The anterior approach involves a surgeon seeking access to the spine through the front (i.e., abdominal area) of the patient. The posterior approach involves a surgeon seeking access to the spine through the back of the patient. The postero-lateral approach is similar to the posterior approach with access coming more from either or both sides of the patient. A variety of different anterior, posterior and posterior-lateral techniques are known.

### SUMMARY OF THE INVENTION

While the invention will be described in connection with certain embodiments, it will be understood that the invention is not limited to these embodiments. On the contrary, the invention includes all alternatives, modifications and equivalents as may be included within the spirit and scope of the present invention.

A spinal implant is provided having at least one piece of cortical bone. The body has a tapered leading end, a trailing end and first and second sides. The body further includes superior and inferior surfaces that are inclined relative to one another. A first plurality of grooves is formed in the superior surface and a second plurality of grooves is formed in the inferior surface. Each of the grooves of the first and second pluralities of grooves include first and second faces converging toward and intersecting one another, and each groove has a maximum cross-sectional width. Each adjacent pair of the grooves of the first and second pluralities of the grooves is separated by a generally planar portion of the superior and inferior surfaces, respectively. Each of the generally planar portions has a width that is equal to or greater than the maximum cross-sectional width of each of the grooves of the respective adjacent pair of the grooves.

In one embodiment, the body is generally ring-shaped with the trailing ends and the sides being convex. In this embodiment, the implant may further include an opening extending completely through the implant between the superior and inferior surfaces.

In another embodiment, the implant is crescent-shaped and includes two pieces of cortical bone secured to one another with at least one mechanical connector. In this embodiment, the leading end, trailing end and one of the sides are convex, while the other side is concave.

In another embodiment, the implant further includes an insert made of cancellous bone, wherein the body may be generally U-shaped and is positioned about at least a portion of the insert. The body and the insert are secured to one another by at least one mechanical connector.

### BRIEF DESCRIPTION OF THE DRAWINGS

The accompanying drawings, which are incorporated in and constitute a part of this specification, illustrate embodiments of the invention and, together with a general description of the invention given above, and a detailed description of the embodiments given below, serve to explain the principles of the invention.

FIG. 1 is a perspective view of a spinal implant according to one embodiment of the present invention;

FIG. 2 is a top view of the implant shown in FIG. 1;

FIG. 3 is a side elevation view of the implant shown in FIGS. 1 and 2;

FIG. 4 is an opposite side elevation view of the implant shown in FIGS. 1-3;

FIG. 5 is an enlarged view of the encircled portion of FIG. 4;

FIG. 6 is an elevation view of the trailing end of the implant shown in FIGS. 1-5;

FIG. 7 is an elevation view of the leading end of the implant shown in FIGS. 1-6;

FIG. 8 is a perspective view of a spinal implant according to another embodiment of the present invention;

FIG. 9 is a top view of the implant shown in FIG. 8;

FIG. 9A is a cross-sectional view taken along line 9A-9A in FIG. 9;

FIG. 10 is a side elevation view of the implant shown in FIGS. 8 and 9;

FIG. 11 is an opposite side elevation view of the implant shown in FIGS. 8-10;

FIG. 12 is an elevation view of the trailing end of the implant shown in FIGS. 8-11;

FIG. 13 is an elevation view of the leading end of the implant shown in FIGS. 8-12;

FIG. 14 is a perspective view of a spinal implant according to another embodiment of the present invention;

FIG. 15 is a top view of the implant shown in FIG. 14;

FIG. 16 is an elevation view of the leading end of the implant shown in FIGS. 14 and 15;

FIG. 17 is an elevation view of the trailing end of the implant shown in FIGS. 14-16; and

FIG. 18 is a side elevation view of the implant shown in FIGS. 14-17.

### DETAILED DESCRIPTION

The present invention is directed to skeletal implants and methods for placing implants between bones desired to be fused. It is preferred for the implants to be used for vertebral/spinal applications such as fusing cervical, thoracic and/or lumbar intervertebral joints. In the case of fusing an intervertebral joint, implants in accordance with the principles of the present invention can be implanted using an anterior, posterior or postero-lateral approach to the patient's vertebrae.

As used herein, an “implant” includes any implant suitable for facilitating fusion between adjacent bones and includes implants prepared from known implant materials including, non-bone material such as titanium, stainless steel, porous tantalum or other metal, bio-glass, calcium phosphate, ceramic, carbon fiber-based polymers, polymeric materials such as PEEK and biodegradable polymers. However, it is preferred for implants in accordance with the principles of the present invention to be derived from natural bone tissue (e.g., allograft and xenograft bone). It is most preferred for implants in accordance with the principles of the present invention to be derived from natural bone such as from a cadaveric allograft bone source. For example, the implants can be derived by cross-sectioning cortical rings from cadaveric allograft long bones such as femur, tibia or fibula bones or from other bone sources such as the illium. Alternatively, the implants can be formed/molded from ground, sintered or composite bone material. Xenograft bones (e.g., from a bovine source) also can be used.

The term “allograft” will be understood to mean a bone implant from a donor transplanted to a genetically dissimilar recipient of the same species. The term “xenograft” will be understood to mean a bone implant from a donor transplanted to a recipient of a different species.

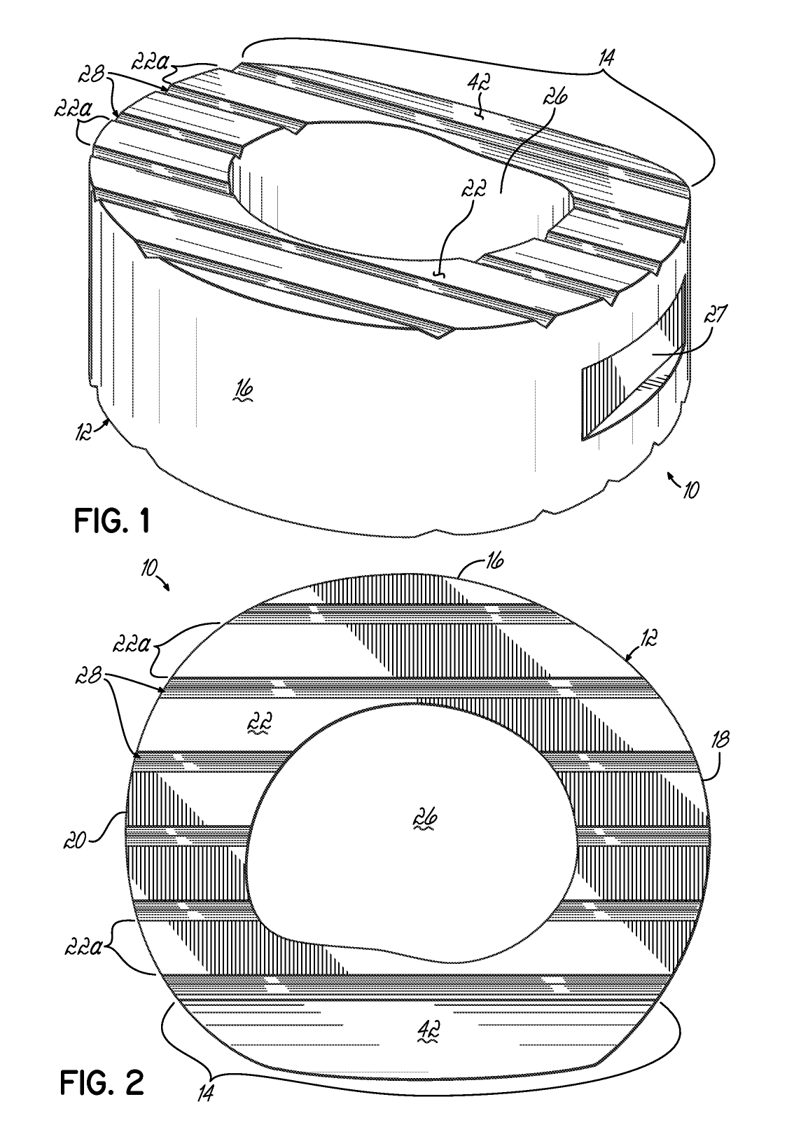

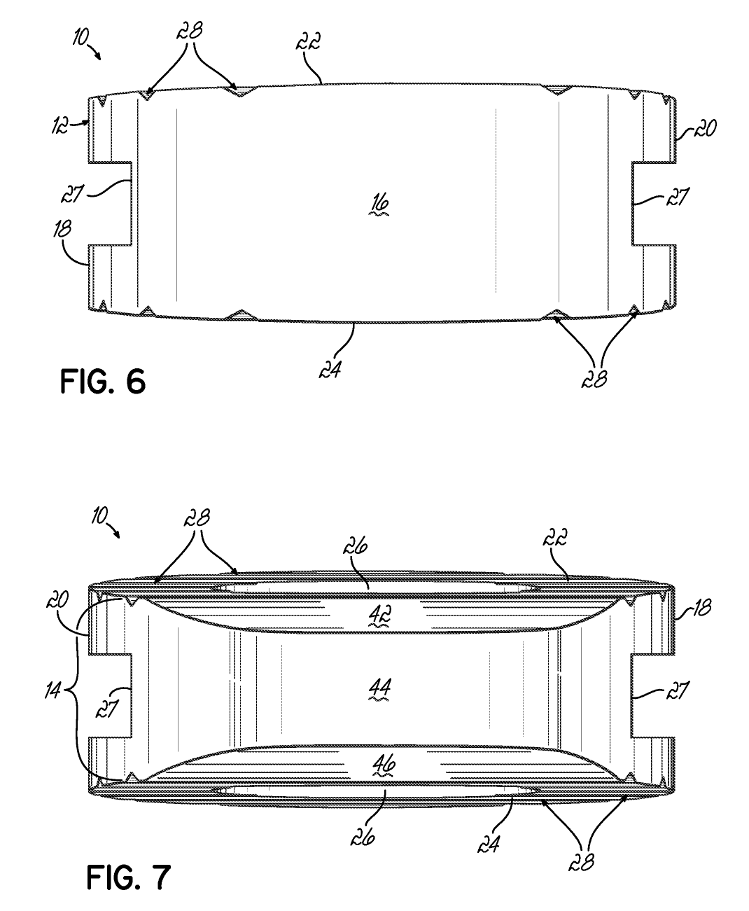

FIGS. 1-7 illustrate an implant 10 according to one embodiment of the present invention. Implant 10 includes a body 12, which is generally ring-shaped as best seen in FIGS. 1 and 2. Body 12 may be made from a single piece of cortical bone. Body 12 includes a tapered leading end 14, a convex trailing end 16, a convex side 18 and an opposite, convex side 20. Body 12 further includes a superior surface 22 and an inferior surface 24. Implant 10 further includes an opening 26 that extends completely through body 12 between the superior 22 and inferior 24 surfaces. In an exemplary embodiment, body 12 may include a pair of indentations 27, one formed in each of the sides 18, 20, for receiving an instrument used to insert implant 10 into a disc space between two adjacent vertebrae.

A first plurality of grooves 28 are formed in the superior surface 22 and a second plurality of the grooves 28 are formed in the inferior surface 24. Grooves 28 facilitate bony ingrowth that promotes fusion of implant 10 to adjacent vertebrae. The grooves 28 formed in the superior surface 22 and the inferior surface 24 extend in a lateral, or side-to-side, direction and may be parallel to one another. As shown in FIG. 2, at least some of the grooves may be interrupted by the opening 26. Referring to FIG. 5, each groove 28 includes first 30 and second 32 faces that converge toward one another, away from the respective superior 22 and inferior 24 surfaces, and intersect one another. Adjacent grooves 28 are spaced apart from one another by a distance “D₁”, that may be the same for grooves 28 formed in the superior surface 22 and the grooves 28 formed in the inferior surface 24.

Referring to FIGS. 1 and 3-5, each adjacent pair of grooves 28 formed in the superior surface 22 is separated by a generally planar portion of the superior surface 22, with one of these generally planar portions designated 22a in FIGS. 4 and 5. Similarly, each adjacent pair of grooves 28 formed in the inferior surface 24 is separated by a generally planar portion of the inferior surface 24, with one of these portions designated 24a in FIG. 4. Each groove 28 has a maximum cross-sectional width “W₁” (FIG. 5) adjacent the generally planar portions of either the superior surface 22 or inferior surface 24. The generally planar portions of the superior 22 and inferior 24 surfaces have a width “W₂”, illustrated with respect to generally planar portion 22a in FIG. 5.

The maximum cross-sectional width “W₁” of each groove 28 on the superior 22 and inferior 24 surfaces may be the same and the spacing “D₁” between each adjacent pair of grooves 28 may be the same. However, it is within the scope of the present invention to have grooves with different widths and different spacings between various adjacent pairs of grooves 28. In either event, the width “W₂” of the substantially planar portions of the superior 22 and inferior 24 surfaces, such as portions 22a, 24a respectively, is selected so that it is greater than or equal to the maximum cross-sectional width “W₁” of each groove 28 of the adjacent pair of grooves 28. For example, width “W₂” of generally planar portion 22a shown in FIG. 5 is selected so that it is equal to or greater than the maximum cross-sectional width “W₁” of groove 28a and is equal to or greater than the maximum cross-sectional width “W₁” of groove 28b. The magnitude of spacing “D₁” may be determined as a result of the particular values of “W₁” and “W₂”. In one exemplary embodiment, “W₁” may have a magnitude ranging from about 0.9 mm to about 1.1 mm and “W₂” may have a magnitude ranging from about 2.65 mm to about 2.85 mm. However, it should be understood that the magnitudes of “W₁” and “W₂” may be different than the foregoing magnitudes in other embodiments. The ratio of width “W₂” to the maximum cross-sectional width “W₁” may have any value equal to or greater than 1 consistent with the overall size of implant 10 and the desired number of grooves 28. In the exemplary embodiment, the ratio of “W₂” to the maximum cross-sectional width “W₁” is greater than 2.

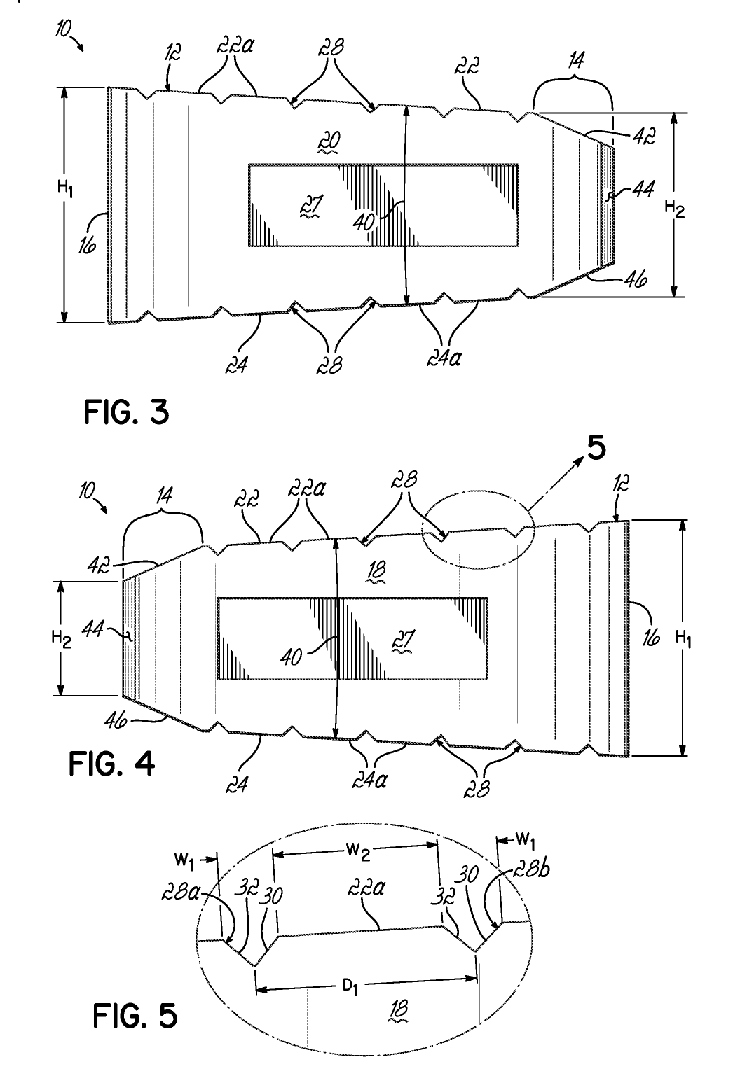

As best seen in FIGS. 3 and 4, the superior 22 and inferior 24 surfaces are inclined relative to one another, with an included angle 40 existing between surfaces 22 and 24. Angle 40 is a lordotic angle. Surfaces 22 and 24 diverge away from one another between the leading 14 and trailing ends 16. Accordingly, a height “H₁” of body 12 at trailing end 16 is greater than a height “H₂” of body 12 at the leading end 14. This configuration accommodates lordosis of the spine. The particular values of “H₁” and “H₂”, as well as the overall width and length of body 12 are consistent with insertion of the implant 10 into the disc space (not shown) of two adjacent vertebrae, such as two adjacent cervical vertebrae in an exemplary embodiment.

Leading end 14 is tapered and includes a first inclined surface 42 that slopes downwardly from the inferior surface 24 to an intermediate surface 44, which is a posterior surface in an exemplary embodiment. Leading end 14 includes a second inclined surface 46, that slopes upwardly from the inferior surface 24 to the intermediate surface 44. The tapered configuration of leading end 14 facilitates insertion of implant 10 into a disc space between adjacent vertebrae. In another embodiment, the superior 22 and inferior 24 surfaces are parallel to one another.

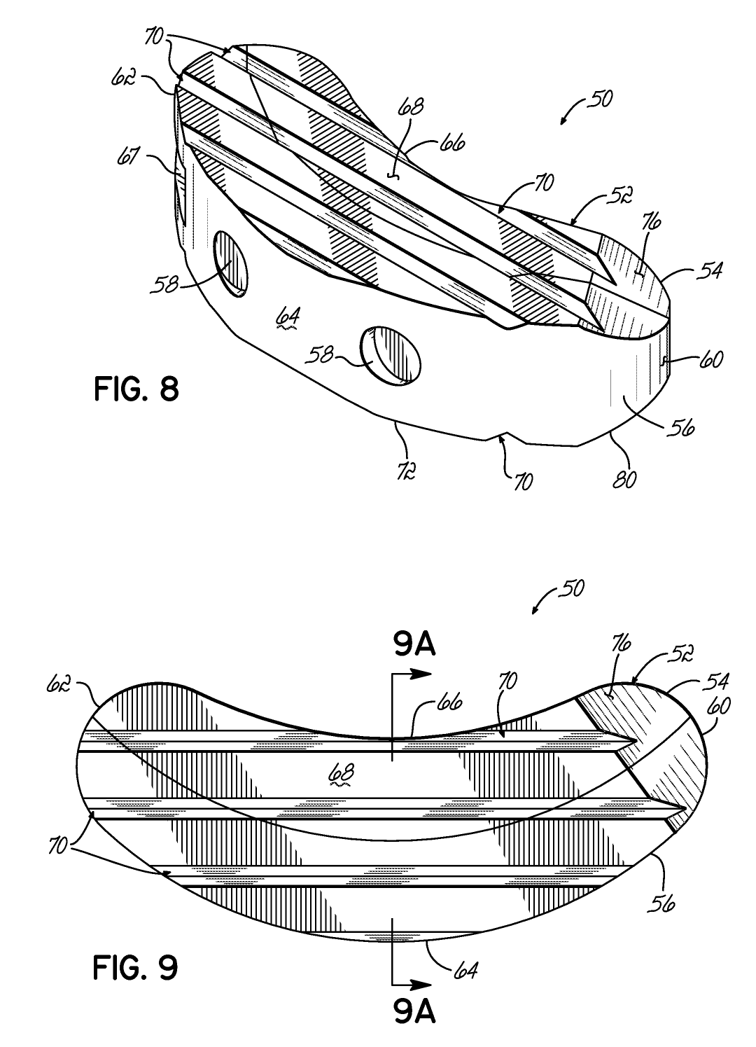

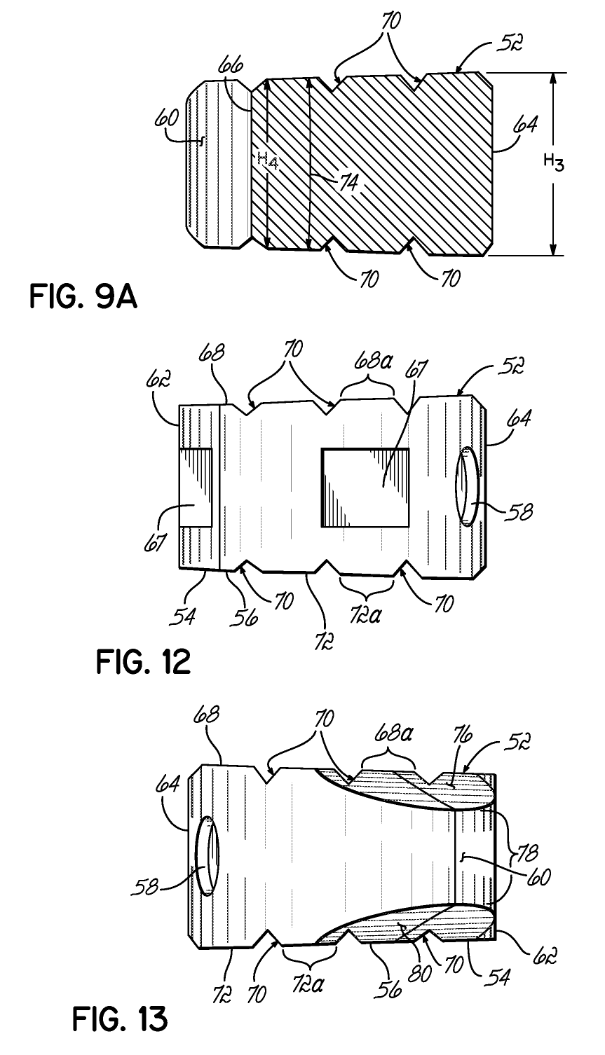

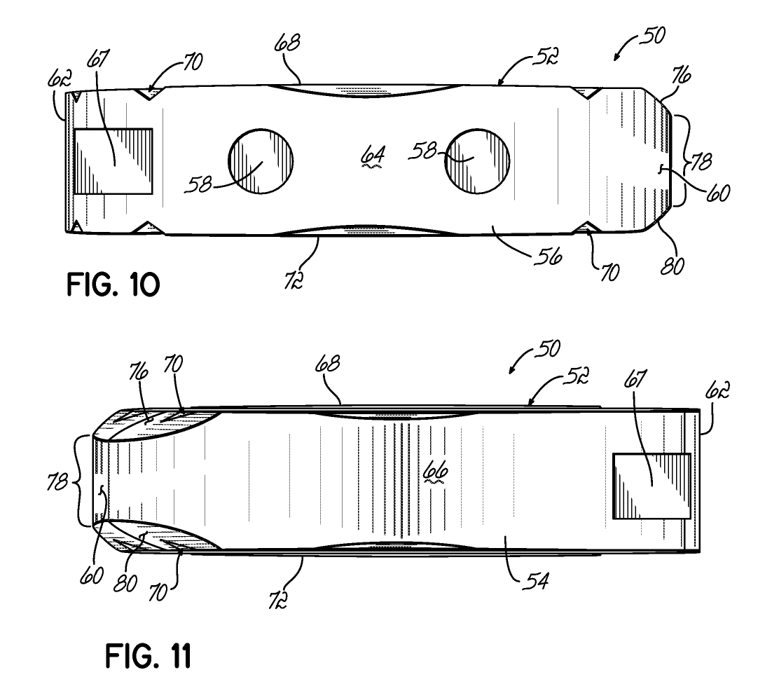

FIGS. 8-13 illustrate a spinal implant 50 according to another embodiment of the present invention. Implant 50 includes a body 52 that may include a first piece 54 of cortical bone and a second piece 56 of cortical bone that may be coupled to one another by one or more mechanical connectors 58, that may be pins, screws or the like, and may be made from cortical bone. Alternatively, the first 54 and second 56 pieces can be coupled to one another with a securing feature on the first 54 and second 56 pieces, such as a dovetail. As may be appreciated with reference to FIGS. 8 and 9, body 52 is crescent-shaped and includes a convex and tapered leading end 60, a convex trailing end 62, a convex side 64 and an opposite concave side 66. In an exemplary embodiment, body 52 may include a pair of indentations 67, one formed in each of the sides 64, 66, for receiving an instrument used to insert implant 50 into a disc space between two adjacent vertebrae.

Body 52 further includes a superior surface 68 and a first plurality of grooves 70 formed therein. A second plurality of the grooves 70 are formed in an inferior surface 72 of body 52. Grooves 70 facilitate bony ingrowth that promotes fusion of implant 50 to adjacent vertebrae. The grooves 70 formed in the superior surface 68 may be parallel to one another and some of the grooves 70 extend between the leading end 60 and the trailing end 62. Similarly, the grooves 70 formed in the inferior surface 72 may be parallel to one another and some of the grooves 70 extend between the leading end 60 and the trailing end 62. Grooves 70 may be shaped and sized the same as grooves 30, of implant 10, and a generally planar portion of superior surface 68, such as portion 68a in FIG. 12, is disposed between each adjacent pair of grooves 70 formed in superior surface 68. Similarly, a generally planar portion of inferior surface 72, such as portion 72a in FIG. 12, is disposed between each adjacent pair of grooves 70 formed in inferior surface 72. Similar to implant 10, the magnitude of the width of each generally planar portion of the superior 68 and inferior 72 surface, such as portions 68a, 72a, respectively, is selected so that it is equal to or greater than the maximum width of each adjacent groove 70. In an exemplary embodiment, the width of each groove 70 and the width of the substantially planar portions of the superior 68 and inferior surfaces 72 may be the same as noted previously for “W₁” and “W₂” of implant 10. In other embodiments, they may be different than the above described and exemplary magnitudes of “W₁” and “W₂”. The spacing between adjacent grooves may be determined as noted previously with regard to spacing “D₁” of grooves 30.

Superior surface 68 and inferior surface 72 are inclined relative to one another, and diverge away from one another between sides 66 and 64 of body 52 defining an included, or lordotic, angle 74 between surfaces 68, 72. Lordotic angle 74 is shown in FIGS. 9A, 12 and 13. Due to the divergence of surfaces 68, 72, side 64 is higher than side 66. For example, at the locations shown in the cross-sectional view illustrated in FIG. 9A, body 52 has a height “H₃” at side 64 that is greater than a height “H₄” of body 52 at side 66. This configuration accommodates lordosis of the spine. The magnitudes of heights “H₃”, “H₄” and included angle 74 may vary with application.

As best seen in FIGS. 11 and 13, the leading end 60 is tapered and includes an inclined surface 76 that slopes downwardly from the superior surface 68 to an intermediate surface 78. A second inclined surface 80 slopes upwardly from the inferior surface 72 to the intermediate surface 78. The tapered configuration of leading end 60 facilitates insertion of implant 50 into a disc space between adjacent vertebrae. In another embodiment, the superior 68 and inferior 72 surfaces are parallel to one another.

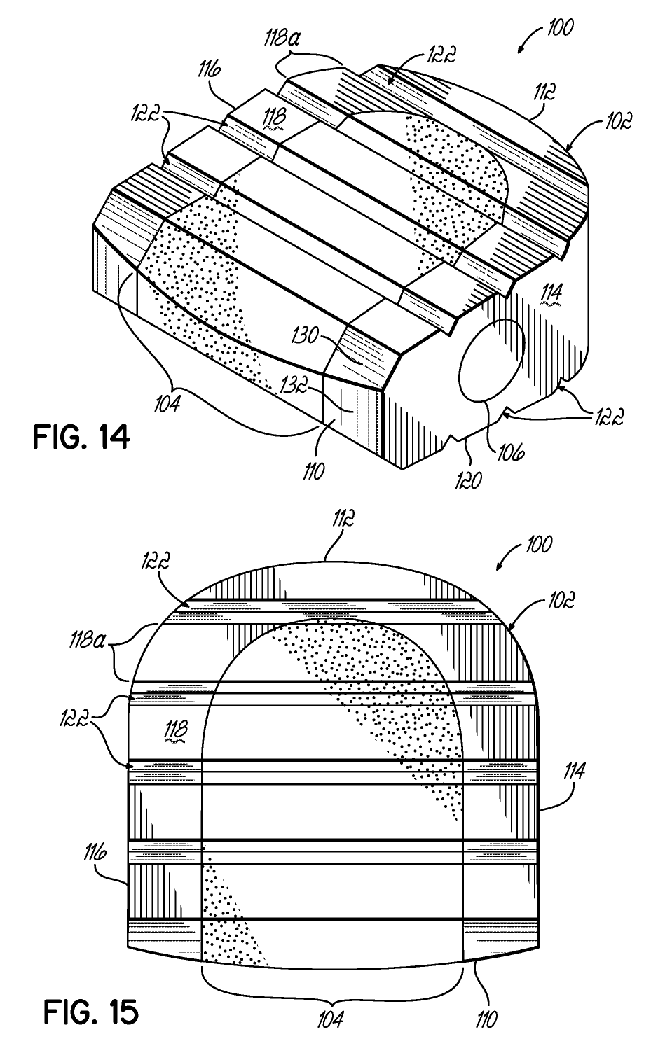

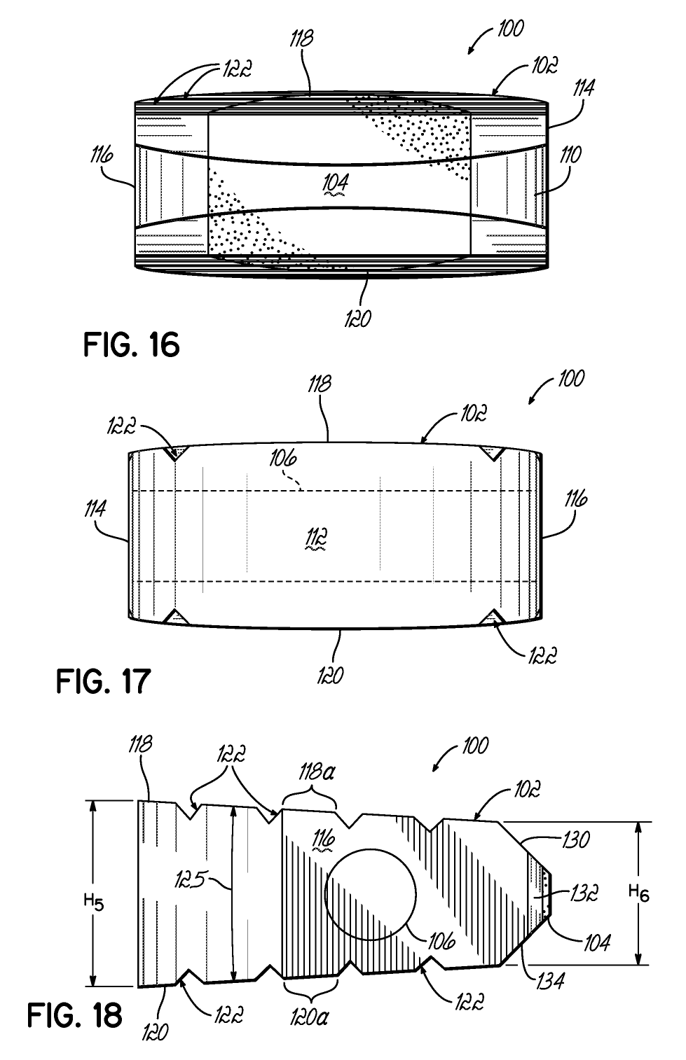

FIGS. 14-18 illustrate a spinal implant 100 according to another embodiment of the present invention. Implant 100 includes a generally U-shaped body 102, which may be made from cortical bone, and an insert 104, which may be made from cancellous bone, having a shape that is complementary to the shape of body 102. As best seen in FIGS. 14 and 15, body 102 is positioned about a portion of insert 104. Body 102 and insert 104 may be secured to one another by at least one mechanical connector 106, such as a pin, screw or the like, which may be made from cortical bone.

Body 102 includes a tapered leading end 110, a trailing end 112 and opposing sides 114, 116. As best seen in FIG. 15, the leading 110 and trailing 112 ends of body 102 are convex. Body 102 further includes a superior surface 118 and an inferior surface 120. A first plurality of grooves 122 are formed in the superior surface 118 and extend from side 114 to side 116 of body 102, with each groove 122 also being formed in a superior surface of insert 104. Similarly, a second plurality of grooves 122 are formed in the inferior surface 120 of body 102 and extend from side 114 to side 116 of body 102, with these grooves also being formed in an inferior surface of insert 104. Grooves 122 facilitate bony ingrowth that promotes fusion of implant 100 to adjacent vertebrae. The grooves formed in the superior surface 118 may be parallel to one another and similarly, the grooves 122 formed in the inferior surface 120 may also be parallel to one another. As best seen in FIGS. 14 and 18, grooves 122 may have the same shape as grooves 30 of implant 10.

A substantially planar portion of the superior 118 and inferior 120 surfaces, such as portion 118a, 120a (FIG. 18), respectively, is disposed between each adjacent pair of grooves 122 formed in the respective superior 118 and inferior 120 surfaces. As with previous embodiments, the magnitude of the width of these generally planar portions, such as portions 118a, 120a, is equal to or greater than the maximum width of each adjacent groove 122. In one exemplary embodiment, the maximum width of each groove 122 may range from about 0.67 mm to about 0.77 mm and the width of the generally planar surfaces between each adjacent pair of grooves 122, such as surfaces 118a, 120a, may range from about 1.39 mm to about 1.49 mm. However, these widths may have different values in other embodiments. The spacing between adjacent grooves 122 may be determined based on the particular magnitudes of the widths of grooves 122 and the generally planar portions of the superior 118 and inferior 120 surfaces.

The superior surface 118 and inferior surface 120 are inclined relative to one another, and diverge away from one another from the leading end 110 to the trailing end 112 of body 102. Accordingly, an included, or lordotic, angle 125 exists between the superior 118 and inferior 120 surfaces. The superior 118 and inferior 120 surfaces of insert 104 are similarly inclined. Accordingly, the body 102 and insert 104, which may be flush with the superior 118 and inferior 120 surfaces of body 102, have a height “H₅” at the trailing end 112 of body 102 that is higher than a height “H₆” at the leading end 110 of body 102. This configuration accommodates the lordosis of the spine. The leading end 110 of body 102 includes an inclined surface 130 that slopes downwardly from the superior surface 118 to an intermediate surface 132. A second inclined surface 134 slopes upwardly from the inferior surface 120 to the intermediate surface 132. The leading end of insert 104 is similarly configured. The configuration of leading end 110 of body 102 and the leading end of insert 104 facilitate insertion of implant 100 into the disc space between adjacent vertebrae, such as between adjacent cervical vertebrae in an exemplary embodiment. In another embodiment, the superior 118 and inferior 120 surfaces are parallel to one another.

The implants 10, 50, 100 can be inserted by a variety of surgical approaches, including, but not limited to an anterior approach, a lateral (transverse) approach, a posterior approach, or postero-lateral approach by engaging the implants 10, 50, 100 with an instrument, such as an inserter. The implants 10, 50, 100 can include grooves, indentations, slots or other surface deficits that allow the inserter to engage the implants 10, 50, 100. For example, the trailing end 16, 62, 112 (of body 102) of the implant can include holes, such as a circular hole or holes that mate with prongs on the inserter. Alternatively, the trailing end 16, 62, 112 (of body 102) can include two or more square or rectangular surface deficits cut into the superior 22, 68, 118 (of body 102) and inferior surfaces 24, 72, 120 (of body 102) that can be engaged by the inserter. In other embodiments, slots or grooves can be formed in each of the sides 18, 64, 114 (of body 102) and 20, 66, 116 (of body 102). The slots or grooves can be partially formed into and engaged at the trailing end 16, 62, 112 (of body 102) by the inserter. The slots or grooves can be formed such that a portion of the implant 10, 50, 100 forms a positive stop for the inserter instrument. Alternatively, the slots or grooves can extend the length of the sides 18, 64, 114 (of body 102) and 20, 66, 116 (of body 102). In an exemplary embodiment, implant 10 includes an indentation 27 formed in each of the sides 18, 20 as shown in FIGS. 1, 3, 4, 6 and 7. In an exemplary embodiment, implant 50 includes an indentation 67 formed in each of the sides 64, 66 as shown in FIGS. 8, 10, 11 and 12.

While the present invention has been illustrated by the description of and exemplary embodiments thereof, and while the embodiments have been described in considerable detail, it is not intended to restrict or in any way limit the scope of the appended claims to such detail. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicants' general inventive concept.

What is claimed is:

1. A spinal implant comprising:
a body comprising at least one piece of cortical bone, said body having a leading end, a trailing end and first and second sides; wherein
said body further includes superior and inferior surfaces, a first plurality of grooves formed in said superior surface and a second plurality of said grooves formed in said inferior surface; wherein
each said groove of said first and second pluralities of said grooves comprises first and second opposing faces converging toward and intersecting one another, each said groove having a maximum cross-sectional width;
each adjacent pair of said grooves of said first and second pluralities of said grooves is separated by a generally planar portion of said superior and inferior surfaces, respectively, each said generally planar portion having a width equal to or greater than said maximum cross-sectional width of each of said grooves of the respective adjacent pair of said grooves.

2. The implant of claim 1, wherein:
said body is generally ring-shaped and said trailing end and said sides are convex;
said implant further comprising an opening extending completely through said body between said superior and inferior surfaces.

3. The implant of claim 1, wherein:
said body is crescent-shaped and comprises two pieces of cortical bone coupled to one another;
said leading and trailing ends of said body are convex, one of said sides is convex and the other of said sides is concave.

4. The implant of claim 1, further comprising:
an insert made of cancellous bone; wherein
said body is generally U-shaped and is positioned about at least a portion of said insert;
said implant further comprises at least one mechanical connector, said body and said insert being secured to one another with said mechanical connector.

5. The implant of claim 1, wherein said superior and inferior surfaces are inclined relative to one another.

6. The implant of claim 1, wherein a ratio of said width of said generally planar portion to said maximum cross-sectional width of said groove is greater than 2.

7. The implant of claim 1, wherein said leading end is tapered.
